(19) United States
(12) Patent Application Publication
(10) Pub. No.: US 2024/0021320 A1
(43) Pub. Date: Jan. 18, 2024

(54) WORKLIST PRIORITIZATION USING NON-PATIENT DATA FOR URGENCY ESTIMATION

(71) Applicant: KONINKLIJKE PHILIPS N.V., EINDHOVEN (NL)

(72) Inventors: NICOLE SCHADEWALDT, NORDERSTEDT (DE); ROLF JÜRGEN WEESE, NORDERSTEDT (DE); MATTHIAS LENGA, MAINZ (DE); AXEL SAALBACH, HAMBURG (DE); STEFFEN RENISCH, HAMBURG (DE); HEINRICH SCHULZ, HAMBURG (DE)

(21) Appl. No.: 18/036,833

(22) PCT Filed: Nov. 11, 2021
(86) PCT No.: PCT/EP2021/081327

§ 371 Date: May 12, 2023

Related U.S. Application Data
(60) Provisional application No. 63/114,741, filed on Nov. 17, 2020.

(51) Int. Cl. G16H 50/70; G16H 10/60; G06N 3/08

(52) U.S. Cl. CPC G16H 50/70; G16H 10/60; G06N 3/08

(57) ABSTRACT

A system and method for training a deep learning network with previously read image studies to provide a prioritized worklist of unread image studies. The method includes collecting training data including a plurality of previously read image studies, each of the previously read image studies including a classification of findings and radiologist-specific data. The method includes training the deep learning neural network with the training data to predict an urgency score for reading of an unread image study.

### BACKGROUND

Radiological examinations including medical image studies such as, for example, X-ray, MRI and CT, are often the most efficient method for diagnosing and/or treating certain conditions. Thus, the number of image studies required to be read at any given time is increasing very rapidly. Due to the large number of image studies required to be read, however, the image studies may be distributed to different departments and/or hospitals for reading and, in some cases, may even be outsourced to a different country so that the radiological reading is disconnected from the data acquisition. While distribution of the image studies may potentially speed the reading of important data, some external prioritization of the image studies is required to optimize workflow.

Some current workflow prioritization systems determine a prioritization based on a simple First In—First Out (FIFO) method, which prioritizes the image studies based on when the image study was acquired and/or received via the radiologist. The FIFO method, however, does not consider a severity of a potential condition to be identified. Some conditions may be time critical so that a speedy review and diagnosis is essential, while some conditions may do well with a multi-day timeframe until report.

In other workflow prioritization systems, workflow prioritization may be determined based on an identified list of potential image classifications (e.g., findings of specific characteristics and/or features in an image). The list of potential image classifications is used to prioritize the image studies based on a hierarchy of conditions—e.g., multiple critical conditions, less critical conditions and normal cases. The hierarchy of conditions, however, does not take into consideration a prioritization within a single classification or between two similarly severe conditions/classifications.

### SUMMARY

The exemplary embodiments are directed to a computer-implemented method of training a deep learning network with previously read image studies to provide a prioritized worklist of unread image studies, comprising: collecting training data including a plurality of previously read image studies, the previously read image studies including a classification of findings and radiologist-specific data; and training the deep learning neural network with the training data to predict an urgency score for reading of an unread image study.

The exemplary embodiments are directed to a system of training a deep learning network with previously read image studies to provide a prioritized worklist of unread image studies, comprising: a non-transitory computer readable storage medium storing an executable program; and a processor executing the executable program to cause the processor to: collect training data including a plurality of previously read image studies, each of the previously read image studies including a classification of findings and radiologist-specific data; and train the deep learning neural network with the training data to predict an urgency score for reading of an unread image study.

The exemplary embodiments are directed to a non-transitory computer-readable storage medium including a set of instructions executable by a processor, the set of instructions, when executed by the processor, causing the processor to perform operations, comprising: collecting training data including a plurality of previously read image studies, each of the previously read image studies including a classification of findings and radiologist-specific data; and training the deep learning neural network with the training data to predict an urgency score for reading of an unread image study.

### BRIEF DESCRIPTION OF THE DRAWINGS

FIG. 1 shows a schematic diagram of a system according to an exemplary embodiment.

FIG. 2 shows another schematic diagram of the system according to FIG. 1.

FIG. 3 shows a flow diagram of a method for deep learning according to an exemplary embodiment.

### DETAILED DESCRIPTION

The exemplary embodiments may be further understood with reference to the following description and the appended drawings, wherein like elements are referred to with the same reference numerals. The exemplary embodiments relate to systems and methods for machine learning and, in particular, relate to systems and methods for training a deep learning neural network to determine an urgency of an image study to be read. The urgency may be used to determine a workflow prioritization and/or to distribute the image study. Exemplary embodiments describe training the neural network to determine the urgency using previously read and reported image studies along with corresponding patient-specific information such as, for example, a patient's age, gender and co-morbidities. The neural network may additionally be trained with radiologist-specific information such as, for example, radiologist expertise and review time.

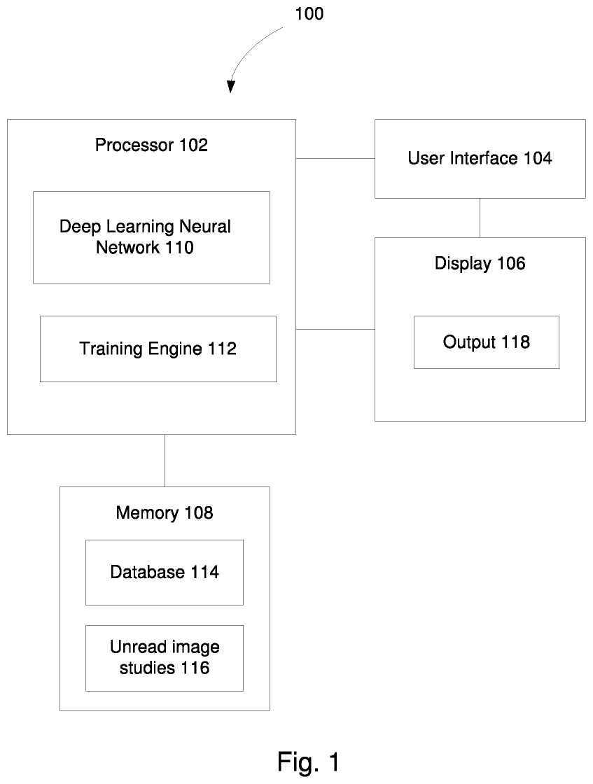

As shown in FIG. 1, a system 100 according to an exemplary embodiment of the present disclosure trains a deep learning neural network 110 to predict or estimate an urgency for a radiological reading of an unread image study. This predicted urgency may then be used to determine a workflow prioritization of a plurality of unread image studies waiting to be read by a radiologist. The system 100 comprises a processor 102, a user interface 104, a display 106 and a memory 108. The processor 102 includes the deep learning neural network 110 and a training engine 112 for training the deep learning neural network 110. The deep learning neural network 110 may be trained using training data stored to a database 114, which may be stored to the memory 108. The training data may include a plurality of previously read image studies in one of a variety of modalities (e.g., X-ray, CT, MRI). Each of the previously read image studies is collected and stored to the database 114 along with relevant patient-specific data (e.g., age, gender, co-morbidities), classification of findings in the image study (e.g., specific features and/or characteristics in an image which, in combination with further information, may be indicative of a condition), diagnoses, and radiologist-specific data (e.g., radiologist specialty or expertise, duration of reading time, tools used for review, priority for treatment).

The processor 102 may be configured to execute computer-executable instructions for operations from applications that provide functionalities to the system 100. For example, the training engine 112 may include instructions for training of the deep learning model 110. It should be noted, however, that functionalities described with respect to the deep learning neural network 110 may also be represented as a separately incorporated component of the system 100, a modular component connected to the processor 102 or as a functionalities achievable via more than one processor 102. For example, the system 100 may be comprised of a network of computing systems, each of which includes one or more of the components described above. It will be understood by those of skill in the art that although the system 100 shows and describes a single deep learning neural network 110, the system 100 may include a plurality of deep learning neural network 110, each learning model trained with training data corresponding to a different image study modality, a different target portion of the patient body and/or a different pathology.

Although the exemplary embodiments show and describe the database 114 of training data as being stored to the memory 108, it will be understood by those of skill in the art that training data may be acquired from any of a plurality of databases stored by any of a plurality of devices connected to and accessible via the system 100 via, for example, a network connection. In one exemplary embodiment, the training data may be acquired from one or more remote and/or network memories and stored to a central memory 108. Alternatively, the training data may be collected and stored to any remote and/or networked memory. Alternatively, the training data for different components of the neural network 110 or different neural networks within the system 110 may be stored on different memories at different institutions, which are no longer accessible by the system 100 so that only the trained networks would be available to the system 100 for the full process or for part of the process (e.g. the finding classification).

Upon completion of an initial training of the deep learning network 110, the deep learning network 110 may be used to determine an urgency for each of a plurality of unread image studies during an inference stage. Urgency may be represented via an urgency score or rate, which is indicative of a level of urgency required for the reading of an unread image study. For example, an urgency score may be on a scale from 0 to 10, with 0 indicating no urgency and indicating an extremely urgent case requiring immediate attention (e.g., a ruptured aneurysm). The urgency scores for each of the unread image studies may be used to generate a prioritized queue of unread image studies for a specific radiologist, department or hospital to which the unread image studies have been distributed and/or assigned. In some embodiments, the urgency score along with other relevant data may be used to determine a distribution of one or more of the unread image studies. The unread image studies may be acquired and received from any of a plurality of imaging devices. It will be understood by those of skill in the art that the imaging devices may transmit the unread image study to the system 100 and/or be in network with the system 100. The unread image studies may similarly be received via the processor 102 and/or stored to the memory 108 or any other memory, remote or in network. The unread image studies may have any of a variety of modalities.

During review of the unread image studies, a worklist prioritization and/or an unread image study may be displayed to a user (e.g., radiologist) on the display 106 of the system 100 or, alternatively, on a display of a computing system in network communication with the system 100. A predicted classification of findings/conditions and/or the predicted urgency of the unread image study and/or additional parameters such as the predicted reading time may also be displayed to the user. In another embodiment, the radiologist may provide his/her own urgency score for the displayed image study via, for example, the user interface 104. The user interface 104 may include any of a variety of input devices such as, for example, a mouse, a keyboard and/or a touch screen via the display 106. This user-provided urgency score and the radiology report may be stored to the database 114 for continuous training of the deep learning neural network 110.

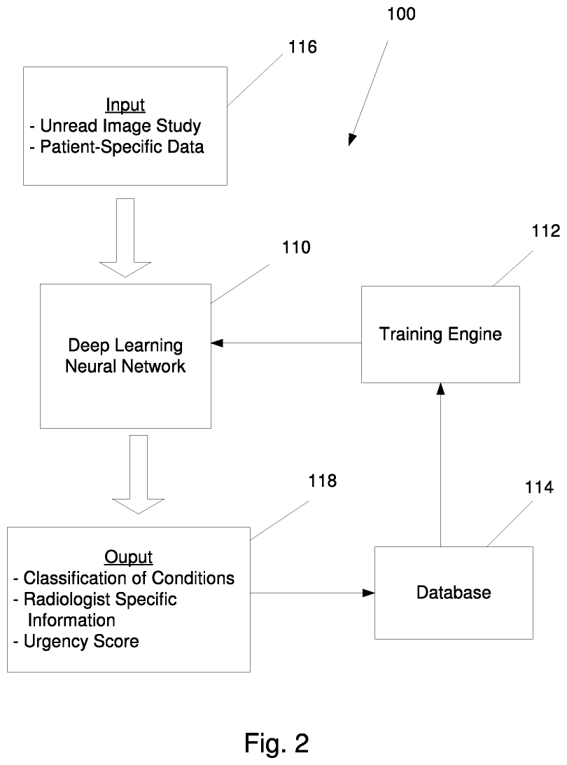

As shown in FIG. 2, according to an exemplary embodiment, the deep learning neural network 110 is trained so that, during the inference stage, when an input 116 including an unread image study and corresponding patient data is directed to the deep learning neural network 110, the deep learning neural network 110 generates an output 118 including an urgency score. In some embodiments, patient-specific data along with features of the unread image study may be used to predict an urgency of the unread image study. In other embodiments, the deep learning neural network 110 may predict both a classification of findings for an image and meta reading parameters (e.g., an estimated review time, an estimated reading time per subspecialty, or whether special tools will be required to be used), along with a secondary prediction urgency. The classification of findings along with the radiological reading prediction may be used to predict an urgency for reading of an image study. For example, severe cases may be recognized immediately and thus have very short review times, while mild cases may be difficult to distinguish from normal images or from other conditions and may require longer review times. In addition, for conditions that may be difficult to distinguish, special tools for image review may be used. Normal cases may have even longer review times as multiple conditions may need to be ruled out. Some urgent conditions may be more easily detected by specialists. For example, rare issues in the lung may be detected faster by lung specialists, but slower by radiologists of other specialties so that the reading time in combination with the specialty may be indicative of both the severity of the condition as well the preferred distribution to a lung specialist.

In further embodiments, in response to the outputted data for the unread image study and/or during a review of the unread image study, the user may provide his/her own urgency score. The user-provided urgency score along with other relevant information such as, for example, a classification of findings for the image study and/or radiologist-specific information may be stored to the database 114. Thus, the training engine 112 uses the database 114 to continuously train the deep learning neural network 110 so that the deep learning neural network 110 may also include user-provided urgency scores. It will be understood by those of skill in the art that user-provided urgency scores should be normalized and carefully defined to ensure consistency between radiologists.

The urgency predicted via the deep learning neural network 110 may be used to provide worklist organization/prioritization, distribution to specific radiologists and/or tool setup or time prediction for a smooth and efficient workflow.

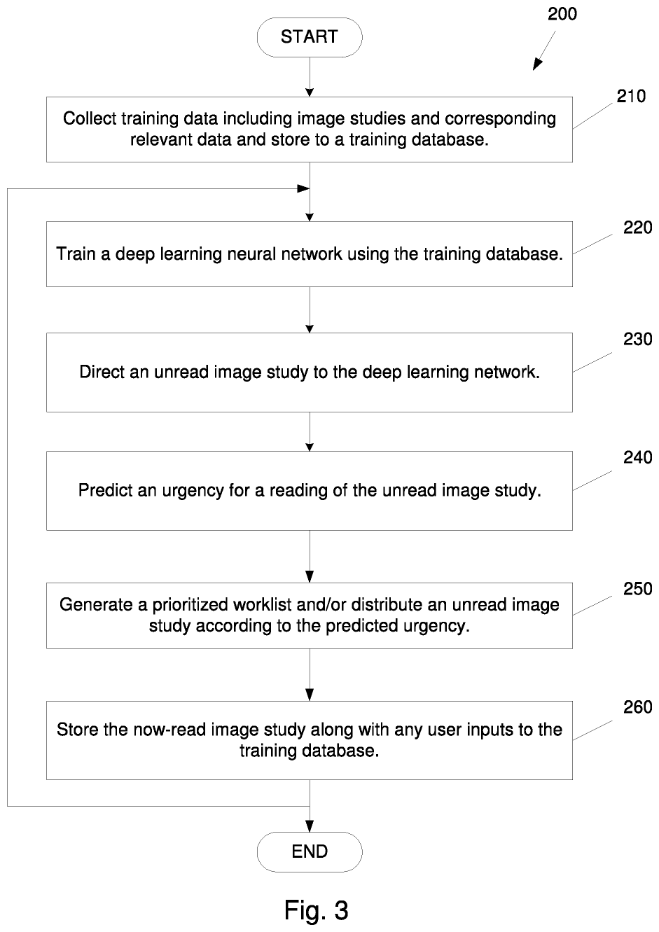

FIG. 3 shows an exemplary method 200 for the deep learning neural network 110 of the system 100. As described above, the deep learning neural network 110 is trained to predict an urgency for the reading of an image study. In 210, training data including previously read/reviewed image studies is collected and stored to the database 114. Each previously read image study includes patient data, a classification of findings and radiologist-specific data. Patient-specific data may include patient identifying information such as, for example, age and gender, along with patient symptoms and/or co-morbidities. The classification of findings may include specific features and/or characteristics in the image study which may be used to identify conditions, illnesses and/or diseases. Radiologist-specific data may include, for example, a duration of review/reading of the image study, a radiologist expertise/specialty, reading time during the day, and use of tools to aid in reading the image study. It will be understood by those of skill in the art that during an initial training of the deep learning neural network 110, the training data may not include urgency values or scores. However, as users (e.g., radiologists) provide their own urgency values to, for example, unread image studies during a review, the database 114 of training data may be updated to include the now read image study along with all corresponding relevant information, including the user-provided urgency score.

In 220, the training engine 114 trains the deep learning neural network 110 with the training data collected in 210. In particular, the training engine 114 trains the deep learning network 110 to be able to predict an urgency (u) of an image study based on an input including the image study (i) and relevant patient-specific information (p) such as, for example, age, gender, symptoms and/or co-morbidities. As discussed above, the urgency may be represented via an urgency score which, for example, may have a quantitative value on a scale from 0 to 10. The deep learning neural network 110 learns each image study of the training data via a convolutional neural network (CNN) including a plurality of convolutional layers applying filters to each of the image studies until a feature map of the image is derived. The feature maps may then be converted to a feature vector, which is followed by a plurality of fully connected layers representative of each of the feature vectors of the feature map.

According to one exemplary embodiment, the deep learning neural network 110 is trained to directly predict the urgency of an image study using the equation:

$$( i , p ) ∈ ( {R}^{2} \to R or {R}^{3} \to R , {R}^{N} ) {\to }^{CNN} ( u ) ∈ ( R )$$

According to another exemplary embodiment, the deep learning neural network 110 is trained to directly predict both a classification of findings (c) and an urgency (u) from a tuple of an image (i) and patient-specific data (p). A classification of the condition or disease is generally considered to be a strong indicator for urgency and thus may partially serve as a control. In this embodiment, the classification ground truth may be used to derive urgency training ground truth. However, as the same condition classification may incorporate more severe and less severe cases, the urgency score may also allow prioritization within the same classification, which is not possible in any currently known embodiments. As this distinction is not possible to derive from available training data, this requires urgency input from experts. However, for some conditions the derivation of an urgency score from the classification may still be useful, e.g. assigning normal cases automatically with urgency 0. The deep learning neural network 110 may be trained to predict classification and urgency using the equation:

$$( i , p ) ∈ ( {R}^{2} \to R or {R}^{3} \to R , {R}^{N} ) {\to }^{CNN} ( c , u ) ∈ ( {R}^{K} , R )$$

According to another exemplary embodiment, the deep learning neural network 110 may be trained to directly predict classification and reading parameters (e.g., duration of reading time, etc.) along with a secondary prediction for urgency. As discussed above, in some embodiments, a user-provided urgency score may be stored to the database to be included in the training data so that the deep learning neural network 110 may be trained to predict the urgency from the predicted classification of findings and radiologist-specific parameters (r)—e.g., prediction of duration of reading time, viewing tools used, radiologist expertise, etc. According to this embodiment, the deep learning network 110 may be trained using the equation:

$$( i , p ) ∈ ( {R}^{2} \to R or {R}^{3} \to R , {R}^{N} ) {\to }^{CNN} ( c , r ) ∈ ( {R}^{K} , {R}^{M} ) {\to }^{f} u ∈ R$$

The urgency for reading of an image, as described above, is an inherently continuous target, which is dependent on both discrete parameters such as a classification of findings (c) and on continuous parameters such as radiologist reading parameters (r) and continuous image input (i). The equation of this embodiment may be particularly useful for determining a worklist prioritization within classifications, distribution to radiologists, viewing tool setup and and/or time prediction for a smooth and efficient workflow. In some embodiment, particularly where multiple parameters (e.g., c and r) are to be predicted, the deep learning neural network 110 may be trained using multi-task learning.

Upon an initial training of the deep learning neural network 110, in 230, an input including an unread image study 116 along with patient-specific data corresponding to the unread image study 116 is directed to the deep learning neural network 110. In 240, the deep learning neural network outputs a prediction of an urgency for the reading of the unread image study using, for example, any of the equations described above with respect to 220. In some embodiments, the predicted urgency may be outputted along with predicted classifications and/or predicted reading parameters.

As discussed above, in 250, the predicted urgency may be used to generate a prioritized worklist within a classification, distribution of the unread image study and/or to optimize workflow. In one embodiment, a user may receive a prioritized worklist including cases prioritized by both classification and urgency. In particular, cases within even the same classification may be prioritized via severity, which would be reflected in the urgency score assuming appropriate successful training. In another embodiment, the urgency predicted in 240 may be used to determine a distribution of a particular unread image study. For example, an unread image study predicted to have a certain classification of findings may be distributed to a radiologist with expertise in that area. In another example, an unread study predicted to have a classification, which is both urgent and differs highly in reading time according to specialty, may be distributed to a radiologist with a specialty that is quick to detect this condition. In another example, an unread image study requiring immediate reading may be distributed to a radiologist who is known to be immediately available. In yet another embodiment, where use of a particular viewing tool is predicted, workflow may be optimized by setting up the viewing tool on the user interface of a radiologist to whom the unread image study has been distributed.

Once a user has reviewed the unread image study, the now-read image study with the user's diagnoses and reading parameters may be stored to the training database 114 for continued training of the deep learning neural network 110. As described above, during review of the unread image study, the user may provide his/her own urgency score, which may also be stored to the training database 114 for training of the deep learning neural network 110. The use of the newly acquired data for a network training may be put on hold for certain conditions, e.g. to wait for confirmation of condition (e.g. from pathology), for outcome of treatment, for tumor board discussion of the finding, or similar, to ensure high quality training data for the network. It will be understood by those of skill in the art that the method 200 may be continuously repeated, as shown in FIG. 3, so that the deep learning network 110 is dynamically expanded and modified with each use thereof. User input may be utilized to continually adapt and modify the deep learning neural network 110.

Those skilled in the art will understand that the above-described exemplary embodiments may be implemented in any number of manners, including, as a separate software module, as a combination of hardware and software, etc. For example, the deep learning neural network 110 and/or the training engine 112 may be a program including lines of code that, when compiled, may be executed on the processor 102.

Although this application described various embodiments each having different features in various combinations, those skilled in the art will understand that any of the features of one embodiment may be combined with the features of the other embodiments in any manner not specifically disclaimed or which is not functionally or logically inconsistent with the operation of the device or the stated functions of the disclosed embodiments.

It will be apparent to those skilled in the art that various modifications may be made to the disclosed exemplary embodiments and methods and alternatives without departing from the spirit or scope of the disclosure. Thus, it is intended that the present disclosure cover the modifications and variations provided that they come within the scope of the appended claims and their equivalents.

1. A computer-implemented method for training a deep learning network with previously read image studies to provide a prioritized worklist of unread image studies, the method comprising:
collecting training data including a plurality of previously read image studies, the previously read image studies including a classification of findings, radiologist-specific data, and patient data, the patient data including one or more of a patient's age, gender, symptoms, and co-morbidities; and
training the deep learning neural network with the training data to predict an urgency score for reading of an unread image study;
wherein the deep learning neural network is trained to predict a classification of findings and radiological reading parameters for the unread image study to derive the urgency score for reading of the unread image study therefrom.

2. The method of claim 1, wherein the radiologist-specific data includes urgency scores for the previously read image studies so that the deep learning neural network is trained to directly predict the urgency score for reading of the unread image study.

3. (canceled)

4. (canceled)

5. The method of claim 1, wherein the radiologist-specific data includes one of a duration of reading time of the previously read image study, a radiologist specialty, and whether a viewing tool was used via the radiologist during a reading of the preciously read image study.

6. The method of claim 1, further comprising:
receiving an unread image study; and
applying the deep learning network to the unread image study to predict an urgency for the reading of the unread image study.

7. The method of claim 6, further comprising:
generating a prioritized worklist for a plurality of unread image studies based on a predicted urgency for each of the plurality of unread image studies.

8. The method of claim 1, further comprising:
distributing each of the unread image studies to one of a plurality of users based on the predicted urgency.

9. The method of claim 8, wherein distributing each of the unread image studies is further based on one of predicted classification of findings and a predicted radiological reading parameters.

10. The method of claim 1, further comprising:
storing results of a reading of the unread image study to a training database for continued training of the deep learning neural network.

11. A system for training a deep learning network with previously read image studies to provide a prioritized worklist of unread image studies, the system comprising:
a non-transitory computer readable storage medium storing an executable program; and
a processor executing the executable program to cause the processor to:
collect training data including a plurality of previously read image studies, the previously read image studies including a classification of findings, radiologist-specific data and patient data, the patient data including one or more of a patient's age, gender, symptoms, and co-morbidities; and
train the deep learning neural network with the training data to predict an urgency score for reading of an unread image study;
wherein the deep learning neural network is trained to predict a classification of findings and radiological reading parameters for the unread image study to derive the urgency score for reading of the unread image study therefrom.

12. The system of claim 11, wherein the radiologist-specific data includes urgency scores for the previously read image studies so that the deep learning neural network is trained to directly predict the urgency score for reading of the unread image study.

13. (canceled)

14. (canceled)

15. The system of claim 11, wherein the radiologist-specific data includes one of a duration of reading time of the previously read image study, a radiologist specialty, and whether a viewing tool was used via the radiologist during a reading of the preciously read image study.

16. The system of claim 11, wherein the processor executes the executable program to cause the processor to:
receive an unread image study; and
apply the deep learning network to the unread image study to predict an urgency for the reading of the unread image study.

17. The system of claim 16, wherein the processor executes the executable program to cause the processor to:
generate a prioritized worklist for a plurality of unread image studies based on a predicted urgency for each of the plurality of unread image studies.

18. The system of claim 11, wherein the processor executes the executable program to cause the processor to:
distribute each of the unread image studies to one of a plurality of users based on the predicted urgency.

19. The system of claim 18, wherein distributing each of the unread image studies is further based on one of predicted classification of findings and a predicted radiological reading parameters.

20. A non-transitory computer-readable storage medium including a set of instructions executable by a processor, the set of instructions, when executed by the processor, causing the processor to perform operations, comprising:
collecting training data including a plurality of previously read image studies, the previously read image studies including a classification of findings, radiologist-specific data and patient data, the patient data including one or more of a patient's age, gender, symptoms, and co-morbidities; and
training the deep learning neural network with the training data to predict an urgency score for reading of an unread image study;
wherein the deep learning neural network is trained to predict a classification of findings and radiological reading parameters for the unread image study to derive the urgency score for reading of the unread image study therefrom.
